(19) United States
(12) Patent Application Publication
(10) Pub. No.: US 2003/0166645 A1
(43) Pub. Date: Sep. 4, 2003

(54) Novel nitrofuran-containing heterocyclic compounds and uses thereof

(76) Inventors: Paul J. Ala, Newark, DE (US); Jacob J. Clement, N.Vancouver (CA); Andrew S. Magee, Maynard, MA (US); Scott T. Moe, Marlborough, MA (US); Manuel A. Navia, Lexington, MA (US); Aloka Roy, Acton, MA (US)

Correspondence Address: FISH & RICHARDSON PC, 225 FRANKLIN ST, BOSTON, MA, 02110, US

(21) Appl. No.: 10/180,806

(22) Filed: Jun. 25, 2002

Related U.S. Application Data
(60) Provisional application No. 60/300,636, filed on Jun. 25, 2001.

(51) Int. Cl. A61K 31/541; A61K 31/5377; A61K 31/496; A61K 31/451; A61K 31/429; A61K 31/4245; A61K 31/4178; C07D 417/02; C07D 413/02; C 07D 4 5/02

(52) U.S. Cl. 514/227.8; 514/235.8; 514/254.07; 514/254.03; 514/326; 514/363; 514/364; 514/383; 514/397; 514/406; 544/60; 544/136; 544/137; 544/366; 544/370; 544/371; 546/210; 546/208; 548/136; 548/143; 548/266.2; 548/315.4; 548/365.7

(57) ABSTRACT

The invention relates to nitrofuran-containing compounds, compositions comprising the compounds, and methods of using the compounds and compound compositions. The compounds and compositions comprising them are useful for treating disease or disease symptoms. The invention also provides for methods of making the compounds, and methods for identifying compounds with desired biological activity.

### CROSS REFERENCE TO RELATED APPLICATIONS

This application claims benefit of U.S. provisional application serial No. 60/300,636, filed Jun. 25, 2001, which is hereby incorporated by reference in its entirety.

### FIELD OF THE INVENTION

This invention relates to heterocyclic compounds and to their use in the medical treatment or prophylaxis of bacterial infections, or their use as antiseptics, sterilizants, or disinfectants.

### BACKGROUND OF THE INVENTION

The pathogenetic process by which microorganisms elicit their adverse effects on hosts is complex and requires a defined sequence of events represented by a variety of microbial components. This process is initiated by the interaction of a host and parasite, typically involving adherence of the microorganism to host surfaces or tissues. Some bacteria (e.g., Bordella pertussis, certain Escherichia coli) attach to mucosal surfaces and exert their ill effects through cell contact and/or proliferation of toxins that further interact with cells. Others (e.g., Salmonella, Shigella) further their effect through tissue penetration and/or cell invasion. The survival and proliferation of the infecting organisms result in toxin production, which, in some instances merely enhances the development of the disease process, while in others, is directly responsible for disease manifestation. If left unchecked, the proliferation of organisms and toxins impairs the host resulting in chronic infection, or even host death. To combat this process, the host has a defense mechanism, however, it is typical, and frequently necessary, to bolster this defense mechanism with exogenous factors, such as antibiotics, to aid clearance of the infecting organism from the host. Over time, however, and due in part to injudicious use of existing antibiotic treatment regimens, organisms are becoming increasingly resistant to the various exogenous factors available.

A need exists for new and effective antibiotic compounds, as the continued effectiveness of existing antibiotics is threatened by rapid development of drug resistance. For example, resistance of bacteria present in urinary tract infections to fluoroquinolones and beta-lactams has been reported and will most probably increase over the next decade. Recent reports have shown that resistance among uropathogens to agents is on the rise. Fluoroquinolone resistance in isolates from community-acquired pneumonia has been increasingly described. Further, there is a serious decrease in susceptibility of E. coli strains to the beta-lactam amoxicillin, due to the presence of R-TEM enzymes (β-lactamases) and cotrimoxazole and trimethoprim. These reports exemplify the necessity and continued need for discovery and development of new antimicrobial therapeutics to provide alternative and more powerful treatment regimens against increasingly resistant microorganisms.

### SUMMARY OF THE INVENTION

The invention relates to heterocyclic compounds, compositions comprising the compounds, and methods of using the compounds and compound compositions. The compounds and compositions comprising them are useful for treating disease or disease symptoms. The invention also provides for methods of making the compounds, methods for identifying compounds with desired biological activity.

The invention is based on the discovery that certain heterocyclic compounds have potent antibacterial activity. Further, the ability of these heterocyclic compounds to cross bacterial cell membranes enhances their suitability for use as antibacterial drugs. Thus, this invention relates to novel heterocyclic compounds and to their use in the medical treatment of disease or their prophylactic use, as antiseptics, sterilizants, or disinfectants. The invention is further based on the discovery that certain nitrofuran-containing heterocyclic compounds, including in general, substituted nitrofuranylheterocycles, have potent antibacterial activity and are useful in the treatment of a variety of human diseases and microbial infections. Several parameters can be used in the selection of compounds for use. The parameters include, but are not limited to, in vitro antibacterial potency and spectrum of activity, physical-chemical properties such as, lipophilicity, solubility, and reduction potential, and the in vitro rate of substrate turnover using nitroreductase enzymes. The pharmacokinetic performance in conjunction with the spectrum of in vitro antibacterial activity indicate that the compounds of the invention are useful in the medical treatment of infected organisms, such as mammals with bacterial or protozoal infections or as antiseptics, sterilizants, or disinfectants.

One embodiment is a compound of the formula:

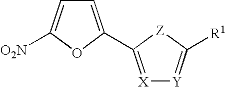

wherein,

Each X is independently selected from CR², NR², O, S, or N;

Each Y is independently selected from CR², NR², O, S, or N;

Each Z is independently selected from CR², NR², O, S, or N;

Each R¹ is independently selected from SH, SO₃H, SO₂NH₂, SO₂NR³R³, S(CH₂)nOH, OR³, S(CH₂)nOC(O)R³, or SR³;

Each n is independently 1, 2 or 3;

Each R² is independently selected from H, C1-C10 alkyl; C2-C10 alkenyl; C2-C10 alkynyl; C3-C10 cycloalkyl; aryl optionally substituted with 1-4 independent R⁵; heteroaryl optionally substituted with 1-4 independent R⁵; heterocyclyl optionally substituted with 1-4 independent R⁵; and C1-C10 alkyl substituted with R⁴ or R⁸;

Each R³ is independently selected from H, C1-C10 alkyl; C2-C10 alkenyl; C2-C10 alkynyl; C3-C10 cycloalkyl; aryl optionally substituted with 1-4 independent R⁵; heteroaryl optionally substituted with 1-4 independent R⁵; heterocyclyl optionally substituted with 1-4 independent R⁵; and C1-C10 alkyl substituted with R⁴ or R⁸;

Each R⁴ is independently selected from halogen, CF₃, SR⁶, OR⁶, OC(O)R⁶, NR⁶R⁶, NR⁶R⁷, COOR⁶, NO₂, CN, C(O)R⁶, or C(O)NR⁶R⁶;

Each R⁵ is independently selected from C1-C10 alkyl; C2-C10 alkenyl; C2-C10 alkynyl; C3-C10 cycloalkyl; C4-C10 cycloalkenyl; halo; haloalkyl; SR⁶; OR⁶; NR⁶R⁶; NR⁶R⁷; COOR⁶; NO₂; CN; C(O)R⁶; C(O)NR⁶R⁶; OC(O)R⁶; S(O)₂R⁶; S(O)₂NR⁶R⁶; NR⁶C(O)N R⁶R⁶; NR⁶C(O)R⁶; NR⁶(COOR⁶); NR⁶C(O)R⁸; NR⁶S(O)₂NR⁶R⁶; NR⁶S(O)₂R⁶; NR⁶S(O)₂R⁸; and C1-C10 alkyl substituted with R⁴ or R⁸;

Each R⁶ is independently selected from H, C1-C10 alkyl; C2-C10 alkenyl; C2-C10 alkynyl; C3-C10 cycloalkyl; R⁸; and C1-C10 alkyl substituted with R⁸ or R¹⁰;

Each R⁷ is independently selected from C(O)R⁹, COOR⁹;

Each R⁸ is independently selected from aryl, heteroaryl or heterocyclyl, each optionally substituted with 1-4 independent R¹⁰;

Each R⁹ is independently C1-C10 alkyl, aryl, heteroaryl, or heterocyclyl, each optionally substituted with 1-4 independent R¹⁰;

Each R¹⁰ is independently halogen, CF₃, SR¹¹, OR¹¹, OC(O)R¹¹, NR¹¹R¹¹, NR¹¹R⁷, COOR¹¹, NO₂, CN, C(O)R¹¹, or C(O)NR¹¹ R¹¹;

Each R¹¹ is independently selected from H, C1-C10 alkyl or aryl, each optionally substituted with halogen, SR¹², OR¹², OC(O)R¹², or NR¹²R¹²;

Each R¹² is independently selected from H, C1-C10 alkyl, heteroaryl, heterocyclyl, or aryl;

Each aryl is independently a 6-carbon monocyclic or 10-carbon bicyclic aromatic ring system wherein 0, 1, 2 or 3 atoms of each ring may be substituted by a substituent;

Each heteroaryl is independently an aromatic 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent; and

Each heterocyclyl is independently a nonaromatic 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent.

In other embodiments, the compound of the formulae herein are those wherein each R¹ is independently selected from SO₃H, SO₂NH₂, SO₂NR³R³, S(CH₂)nOH, or S(CH₂)nOC(O)R³; or alternatively those wherein each R¹ is independently selected from SO₃H, SO₂NH₂, or SO₂NR³R³.

Another embodiment is the compound of any of the formulae herein wherein,

Each Z is independently NR²;

Each R² is independently selected from C2-C10 alkyl; C2-C10 alkenyl; C2-C10 alkynyl; C3-C10 cycloalkyl; aryl optionally substituted with 1-4 independent R⁵; heteroaryl optionally substituted with 1-4 independent R⁵; heterocyclyl optionally substituted with 1-4 independent R⁵; or C1-C10 alkyl substituted with R⁴ or R⁸; and

Each R⁴ is independently selected from halogen, CF₃, SR⁶, OR⁶, OC(O)R⁶, NR⁶R⁶, NR⁶R⁷, NO₂, CN, C(O)R⁶, or C(O)NR⁶R⁶.

In other embodiments, the compound of any of the formulae herein wherein,

Each Z is independently NR²; and

Each R² is independently aryl optionally substituted with 1-4 independent R⁵; heteroaryl optionally substituted with 1-4 independent R⁵; or heterocyclyl optionally substituted with 1-4 independent R⁵; alternatively wherein,

Each Z is independently NR²;

Each R² is independently selected from C1-C10 alkyl substituted with R⁴ or R⁸; and

Each R⁴ is independently selected from halogen, CF₃, SR⁶, OR⁶, OC(O)R⁶, NR⁶R⁶, NR⁶R⁷, NO₂, CN, C(O)R⁶, or C(O)NR⁶R⁶; alternatively wherein,

Each Z is independently NR²; and

Each R² is independently aryl optionally substituted with 1-4 independent R⁵.

Other embodiments are a compound of any of the formulae herein wherein, X and Y are both N; or alternatively wherein, X and Y are not identical, or alternatively wherein X and Y are one of the following: X is CR² and Y is N; or X is N and Y is CR².

Other embodiments are a compound of any of the formulae herein wherein, X, Y and Z are one of the following:

X is CR²,Y is N, and Z is NR²;

X is N, Y is CR², and Z is NR²;

X is N, Y is N, and Z is NR²;

X is CR², Y is N, and Z is O;

X is N, Y is CR², and Z is O;

X is CR², Y is N, and Z is S; or

X is N, Y is CR², and Z is S.

Other embodiments are a compound of any of the formulae herein wherein,

Each R² is independently selected from aryl optionally substituted with 1-4 independent R⁵; heteroaryl optionally substituted with 1-4 independent R⁵; heterocyclyl optionally substituted with 1-4 independent R⁵; and C1-C10 alkyl substituted with R⁴ or R⁸; alternatively wherein, each R² is independently selected from aryl optionally substituted with 1-4 independent R⁵; or heteroaryl optionally substituted with 1-4 independent R⁵.

Other embodiments are a composition comprising a compound of any of the formulae herein and a pharmaceutically acceptable carrier;

a formulation comprising a compound of any of the formulae herein and an excipient suitable for administration to a subject;

a composition comprising a compound of any of the formulae herein, an additional therapeutic agent, and a pharmaceutically acceptable carrier;

a composition comprising a compound of any of the formulae herein, an additional therapeutic agent, and a pharmaceutically acceptable carrier, wherein the additional therapeutic agent is an antibacterial agent;

a method of treating a subject (including a subject in need of such treatment, or identified as in need of such treatment) (e.g., mammal, human, horse, dog, cat, fish, or reptile) infected with one or more bacteria or having a microbial or mycobacterial infection (including, but not limited to urinary tract infections, systemic and topical infections, sepsis, antibiotic mediated colitis, ulcers of the gastrointestinal tract, topical disinfectant, antiseptic, sterilizant, wound care, opportunistic infections in immuno-compromised patients, and surgical cleansing), comprising administering to the subject an effective amount of a compound of any of the formulae herein;

a method for treating infection in a subject (including a subject in need of such treatment, or identified as in need of such treatment) (e.g., mammal, human, horse, dog, cat, fish, or reptile) comprising administration of any of the compositions described herein;

a method of making a compound of any of the formulae herein comprising taking a 5-nitrofuran-2-carbonylchloride and reacting it with one or more chemical reagents in one or more steps to produce a compound of any of the formulae herein;

a method of making a compound of any of the formulae herein comprising taking any one of the intermediate compounds described herein and reacting it with one or chemical reagents in one or more steps to produce a compound of any of the formulae herein;

a method for identifying a compound having antibacterial activity comprising: a) assessing the structure of a compound of any of the formulae herein; b) procuring a derivative compound of the compound in step a); and c) assessing the antibacterial activity of the derivative compound; and

a method for identifying a compound having antibacterial activity comprising: taking a candidate compound; assessing the binding affinity of the candidate compound in a model of the nitroreductase enzyme NFSA; and assessing the antibacterial activity of the candidate compound.

Another embodiment is a compound of any of the formulae herein having one or more of the following properties:

a compound in which the calculated or experimentally determined lipophilicity (logP) is in the range of 0 to 2 logP units;

a compound which is a substrate for any nitroreductase enzyme;

a compound having a redox potential in a suitable range such that the compound is a substrate for the nitroreductase enzyme; the literature values indicate that this range would be approximately between about −0.6 and about −0.2, inclusive E₇¹(V);

a compound having aqueous solubility greater than 1 μg/mL.

Another embodiment is a method of inhibiting bacterial growth in a non-living system comprising contacting the system with an effective amount of a compound of any of formulae herein. The invention features a method of inhibiting bacterial growth in a non-living system (e.g., sterilizing, disinfecting, killing bacteria in vitro). The method includes the step of contacting the system (e.g., a medium, a medical device, a kitchen or bathroom surface, an operating theater), with an effective amount of any of the compounds of the formulae herein, to inhibit bacterial growth.

In another embodiment, the invention is a method of treating bacterial infection in a subject by nitroreductase enzymatic activation of a nitrofuran-containing compound or composition delineated herein comprising administration of the nitrofuran-containing compound or composition to a subject having the nitroreductase enzyme present in the infecting organism. Alternatively, an embodiment is a method of killing bacteria by nitroreductase enzyme activation in a subject (e.g., human, or mammalian or non-mammalian animal) comprising administering to the subject a compound or composition delineated herein. The compound or composition includes an effective amount of compound to result in (after enzyme activation, e.g., reduction or oxidation) killing of bacteria (e.g., bactericidal activity). As such, the compounds herein are useful to treat nitroreductase-mediated disease or disease symptoms, however, the activity of the compounds is not limited to, nor exclusively dictated by, their nitroreductase enzyme activation activity.

The terms “halo” and “halogen” refer to any radical of fluorine, chlorine, bromine or iodine. The terms “alkyl”, “alkenyl” and “alkynyl” refer to hydrocarbon chains that may be straight-chain or branched-chain, containing the indicated number of carbon atoms. For example, C1-C10 indicates the group may have from 1 to 10 (inclusive) carbon atoms in it. The terms “ring” and “ring system” refer to a ring comprising the delineated number of atoms, said atoms being carbon or, where indicated, a heteroatom such as nitrogen, oxygen or sulfur. The ring itself, as well as any substituents thereon, may be attached at any atom that allows a stable compound to be formed.

The term “aryl” refers to a 6-carbon monocyclic or 10-carbon bicyclic aromatic ring system wherein 0, 1, 2 or 3 atoms of each ring may be substituted by a substituent. Examples of aryl groups include phenyl, naphthyl and the like.

The term “heteroaryl” refers to an aromatic 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heterocyclyl” refers to a nonaromatic 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent. Examples of heterocyclyl groups include piperizinyl, pyrrolidinyl, dioxanyl, morpholinyl, tetrahydrofuranyl, and the like.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject or antiseptic, wound dressing impregnation, sterilizant, or disinfectant applications).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The invention may provide several advantages over the existing methods of treatment. For example, the compounds of the invention may have several chemical and pharmacological advantages useful in treating microbial and bacterial infections. These advantages may include both chemical stability and pharmacological stability, as well as potency, different resistance profiles, different selectivity profiles, and decreased side-effects. The invention also envisions veterinary uses for the treatment of infections in fish (e.g., catfish, salmon, trout, goldfish, etc.), fowl (e.g., poultry, chicken, turkey, duck, geese), livestock and agricultural animals (e.g., cows, bulls, pigs, lamb), sports animals (e.g., horses, dogs), ornamental animals (e.g., rabbits, mice, rats, birds, reptiles, goldfish, snakes, lizards), and companion animals (e.g., dogs, cats). Thus, a subject as described herein includes these animals as well as humans.

Other features and advantages of the invention will be apparent from the following detailed description, and from the claims.

### BRIEF DESCRIPTION OF THE FIGURES

FIG. 1 illustrates a model of the E. coli NfsA nitroreductase that activates heterocyclic antibiotics.

FIG. 2 illustrates a close-up view of the NfsA nitroreductase model active site, showing the flavin mononucleotide (FMN) cofactor that mediates heterocycle nitroreduction.

### DETAILED DESCRIPTION OF THE INVENTION

The invention also relates to the specific compounds exemplified herein. Thus one embodiment of the invention is any compound specifically delineated herein, including the compounds listed below:

TABLE 1
Compounds

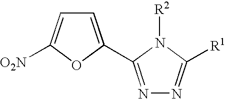

| Compd | R¹ | R² |
|---|---|---|
| 101 | SH | β-D-glucose |
| 102 | SO3H | NH2 |
| 103 | SO₂NHNH₂ | CH₃ |
| 104 | SO₂NH₂ | CH₃ |
| 105 | SO₂NHCH(CH₃)COOH | CH₃ |
| 106 | SO₂NHCH₃ | CH₃ |
| 107 | SO₃H | CH₃ |
| 108 | SOCH₃ | CH₃ |
| 109 | SO₂CH₃ | CH₃ |
| 110 | SH | NH₂ |
| 111 | SH | per-Ac-β-D-glucose |
| 112 | OH | CH₃ |
| 113 | OCOCH₃ | CH₃ |
| 114 | SO₂NHCH₂CH₂CH₃ | CH₃ |
| 115 | OCH₃ | CH₃ |
| 116 | SCH₂CH₂CH₂OH | CH₃ |
| 117 | SH | H |
| 118 | SH | CH₂CH₂N(CH₂CH₂)₂O |
| 119 | SHCH₂CH₂COOH | CH₃ |
| 120 | SH | CH₂CH₂CH₂N(CH₂CH₂)₂O |
| 121 | SCH₂CN | CH₃ |
| 122 | SH | CH₃ |
| 123 | SO₂NHC₆H₅ | CH₃ |
| 124 | SCH₃ | CH₃ |
| 125 | SH | CH₂-2-furanyl |
| 126 | SH | CH₂CH₃ |
| 127 | SO3H | C6H5 |
| 128 | SO3H | CH2C6H5 |
| 129 | SCH₂-4-(C₅H₄N) | CH₃ |
| 130 | SH | CH₂CH═CH₂ |
| 131 | SO3H | C6H11 |
| 132 | SO3H | C6H4-p-Cl |
| 133 | SH | CH₂CH₂CH₂CH₃ |
| 134 | SH | C₆H₅ |
| 135 | SH | CH₂C₆H₅ |
| 136 | SH | 4-F—C₆H₄ |
| 137 | SH | C₆H₁₁ |
| 138 | SH | 4-I—C₆H₄ |
| 139 | SH | 4-Cl—C₆H₄ |
| 140 | SH | 3-Cl—C₆H₄ |
| 141 | SH | 4-Br—C₆H₄ |
| 142 | SH | 3-Cl-4-Br—C₆H₄ |
| 143 | SH | C6H4-p-O—C6H5 |
| 144 | SO2NH(CH2)2OH | CH3 |
| 145 | SO2NHCH(CH3)COOH | CH3 |
| 146 | SO2NH(CH2)2NH2 | CH3 |
| 147 | S(CH2)2OH | CH3 |
| 148 | S(CH2)4OH | CH3 |
| 149 | SCH2COOCH3 | CH3 |
| 150 | SCH2CH2OH | C6H4-p-Cl |
| 151 | SH | C6H4-p-CH3 |
| 152 | SH | C6H4-p-COOC2H5 |
| 153 | SH | C6H4-p-OCH3 |
| 154 | SO2NH(CH2)2OH | C6H4-p-F |
| 155 | SO2NH2 | C6H5 |
| 156 | SO2NH2 | CH2C6H5 |
| 157 | SO2NH2 | C6H5-p-F |
| 158 | SO2NHCH3 | C2H5 |
| 159 | SO2NHCH3 | C4H7 |
| 160 | SO2NHCH3 | C6H5 |
| 161 | SO3H | C2H5 |
| 162 | SO3H | C4H7 |
| 163 | SO3H | C6H5 |
| 164 | OH | C2H5 |
| 165 | OH | CH2CH═CH2 |
| 166 | SO2NHC3H7 | C2H5 |
| 167 | SO2NHC3H7 | CH2CH═CH2 |
| 168 | SCH2CH2OH | CH2-2-furyl |
| 169 | SCH2CH2OH | C2H5 |
| 170 | S(CH2)3OH | CH2-2-furyl |
| 171 | S(CH2)3OH | C2H5 |
| 172 | SCH2CN | CH2-2-furyl |

The compounds of this invention may be synthesized using conventional techniques. Advantageously, these compounds are conveniently synthesized from readily available starting materials. In general, the compounds of the formulae described herein are conveniently obtained via methods illustrated in the schemes and the Examples herein.

Thus, one embodiment relates to a method of making a compound of the formulae described herein, comprising synthesizing any one or more intermediates illustrated in the synthetic schemes herein and then converting that intermediate(s) to a compound of the formulae described herein. Another embodiment relates to a method of making a compound of the formulae described herein, comprising synthesizing any one or more intermediates illustrated in the examples herein and then converting that intermediate(s) to a compound of the formulae described herein. Another embodiment relates to a method of making a compound of the formulae described herein, comprising synthesizing any one or more intermediates illustrated in the synthetic schemes herein and then converting that intermediate(s) to a compound of the formulae described herein utilizing one or more of the chemical reactions described in the synthetic schemes or examples herein. Nucleophilic agents are known in the art and are described in the chemical texts and treatises referred to herein. The chemicals used in the aforementioned methods may include, for example, solvents, reagents, catalysts, protecting group and deprotecting group reagents and the like. The methods described above may also additionally comprise steps, either before or after the steps described specifically herein, to add or remove suitable protecting groups in order to ultimately allow synthesis of the compound of the formulae described herein.

As can be appreciated by the skilled artisan, the synthetic schemes herein are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995); and subsequent editions thereof.

Synthesis of Nitrofuran-Containing Triazole Compounds of the Invention

In general, triazoles (5) can be prepared according to the following procedure: A solution of 5-nitrofuran-2-carbonylchloride (1) in dry methylene chloride or other dry nonpolar solvent is added dropwise to a stirred solution of semicarbazide (2) in dry methylene chloride or other non-polar solvent under an inert atmosphere at a relatively low temperature (e.g., around 0° C.).

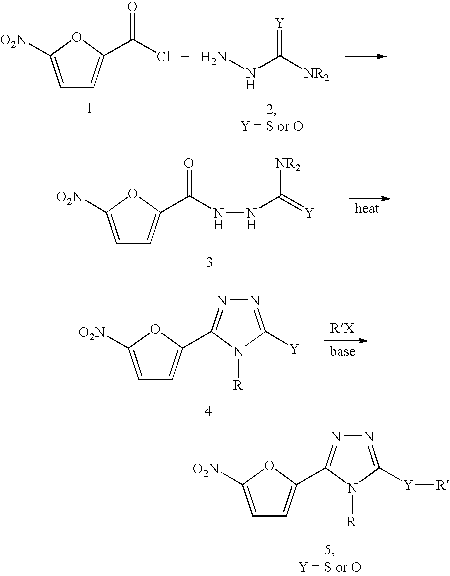

The reaction mixture (containing 3) is slowly warmed to room temperature or higher (e.g., 25° C., 40° C., 80° C., 100° C., or higher) and stirred for 1 to 12 hours or more (e.g., overnight). The reaction mixture is then filtered. The solvent is removed from the mother liquor, and the crude product (4) is purified (e.g., by flash chromatography or recrystallization). Optionally, product 4 can be treated with a base and an alkylating agent (e.g., an alkyl halide) to form an ether or thioether, or with other reagents to modify substituents R or R′. The resulting products (5) can then be purified again.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺salts. This invention also envisions the quaternization of any basic nitrogen-containing groups of the compounds disclosed herein. Water or oil-soluble or dispersible products may be obtained by such quaternization.

In vitro Assays for Antibacterial Activity

The compounds can be screened for antibacterial activity using standard methods.

In one example, illustrated below, broth microdilution techniques are used to measure in vitro activity of the compounds against a given bacterial culture, to yield minimum inhibitory concentration (MIC) data.

Microdilution Antimicrobial Susceptibility Test Assay

Stock solutions of tested compounds were prepared in N,N-dimethylformamide (DMF) at a concentration of 5 mg/mL. Working solutions of the tested compounds were then prepared from the stock solutions, in Mueller-Hinton broth (MHB) with a starting concentration of 64 μg/mL.

Bacterial inocula were prepared from overnight culture (i.e., one fresh colony from agar plate in 5 ml MHB; H. influenzae was grown in MHB with the addition of yeast extract, haematin, and NAD), centrifuged 2×5 min/3000 rpm (for S. pneumoniae and H. influenzae, 2×10 min/3000 rpm), and dispensed in 5 ml of fresh MHB each time, such that the bacterial suspension is diluted to obtain 100 colony forming units (cfu) in a microplate well (100 μl total volume).

Microplate wells were filled with two-fold dilutions of test compound (50 Al), starting with 64 μg/ml. Wells were then filled with 50 μl of bacterial inoculum (final volume: 100 μl/well). The plates were incubated at 37° C. for 18-24 hours (S. pneumoniae was grown in a CO₂-enriched atmosphere).

The optical density of each well at 590 nm (OD₅₉₀) was then measured with a TECAN SpectroFluor Plus@, and minimum inhibitory concentration (MIC) was defined as the concentration that showed 90% inhibition of growth.

Table 2 summarizes MIC values determined for several compounds representative of the invention.

TABLE 2
In vitro antimicrobial activity (MIC) reported in μg/mL for substituted triazoles.

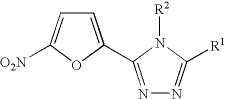

| | | | E coli | E coli (ImpA) | S aureus | E faecalis | S pneumoniae | M. catarrhalis | H influenzae | S cerevisiae | P aeruginosa | |
|---|---|---|---|---|---|---|---|---|---|---|---|---|
| Compd | R¹ | R² | MIC2 | MIC9 | MIC2 | MIC2 | MIC6 | MIC2 | MIC4 | MIC7 | MIC2 | AlogP98 |
| 106 | SO₂NHCH₃ | CH₃ | 8 | 4 | >64 | >64 | 64 | 2 | 32 | 64 | >64 | 0.19 |
| 107 | SO₃H | CH₃ | 2 | 4 | 16 | >64 | 32 | 1 | 16 | 64 | >64 | 0.45 |
| 110 | SH | NH₂ | 16 | 8 | 32 | >128 | 32 | 32 | 8 | 64 | | 0.94 |
| 116 | SCH₂CH₂CH₂OH | CH₃ | 8 | 1 | 8 | 8 | 8 | 0.5 | 16 | >64 | | 1.35 |
| 117 | SH | H | >64 | 16 | 64 | 32 | 2 | 2 | 8 | >64 | | 1.43 |
| 120 | SH | CH₂CH₂CH₂N(CH₂CH₂)₂O | >64 | 32 | 64 | 64 | 16 | 2 | 16 | 64 | | 1.53 |
| 121 | SCH₂CN | CH₃ | 8 | 2 | 16 | 64 | 16 | 1 | 16 | >64 | | 1.55 |
| 122 | SH | CH₃ | 4 | 0.25 | 2 | 2 | 4 | <0.12 | 8 | 32 | | 1.64 |
| 123 | SO₂NHC₆H₅ | CH₃ | >64 | 16 | 64 | 64 | 8 | 1 | 16 | >64 | >64 | 1.77 |
| 124 | SCH₃ | CH₃ | 64 | 2 | 16 | 32 | 8 | 1 | 8 | 16 | | 1.82 |
| 125 | SH | CH₂-2-furanyl | >64 | 4 | 8 | 64 | 8 | 0.5 | 4 | >64 | >64 | 1.85 |
| 126 | SH | CH₂CH₃ | >64 | 64 | >64 | 64 | 16 | 4 | 32 | >64 | | 1.99 |
| 127 | SO3H | C6H5 | >64 | 64 | >64 | >64 | >64 | 32 | >64 | >64 | >64 | 2.03 |
| 128 | SO3H | CH2C6H5 | >64 | >64 | >64 | >64 | 64 | 16 | 64 | >64 | >64 | 2.03 |
| 129 | SCH₂-4-(C₅H₄N) | CH₃ | 64 | 4 | 16 | 64 | 16 | 0.5 | 16 | >64 | | 2.26 |
| 130 | SH | CH₂CH═CH₂ | >64 | 64 | >64 | 64 | 16 | 4 | 32 | >64 | | 2.26 |
| 131 | SO3H | C6H11 | >64 | >64 | >64 | >64 | 64 | 8 | 64 | >64 | >64 | 2.31 |
| 132 | SO3H | C6H4-p-Cl | >64 | 16 | 32 | >64 | 32 | 4 | 8 | 64 | >64 | 2.69 |
| 133 | SH | CH₂CH₂CH₂CH₃ | >64 | 64 | 64 | >64 | 64 | 32 | 32 | 64 | | 2.97 |
| 134 | SH | C₆H₅ | | 2 | 2 | 64 | 8 | 0.25 | 8 | >64 | | 3.22 |
| 135 | SH | CH₂C₆H₅ | >64 | 32 | 32 | 16 | 8 | 1 | 64 | >64 | | 3.22 |
| 136 | SH | 4-F—C₆H₄ | >64 | 16 | 32 | 64 | 8 | 4 | 16 | 64 | | 3.42 |
| 137 | SH | C₆H₁₁ | >64 | 32 | >64 | 32 | 8 | 1 | 16 | >64 | | 3.50 |
| 138 | SH | 4-I—C₆H₄ | >64 | 0.25 | 0.5 | 1 | 0 5 | <0.12 | 8 | 16 | | 3.80 |
| 139 | SH | 4-Cl—C₆H₄ | >64 | <0.12 | 0.25 | 2 | 2 | <0.12 | 2 | 32 | | 3.88 |
| 140 | SH | 3-Cl—C₆H₄ | >64 | 0.5 | 1 | 16 | 2 | <0.12 | 4 | >64 | >64 | 3.88 |
| 141 | SH | 4-Br—C₆H₄ | >64 | <0.12 | 0.25 | 2 | 2 | 0.125 | 8 | 64 | | 3.97 |
| 142 | SH | 3-Cl-4-Br—C₆H₄ | >64 | <0.12 | <0.12 | 1 | 2 | <0.12 | 8 | 64 | | 4.63 |
| 143 | SH | C6H4-p-O—C6H5 | >64 | <0.12 | 0.25 | 1 | 0.5 | <0.12 | >64 | >64 | >64 | |
| | | Trimethoprim | 0.25 | <0.12 | 2 | <0.12 | 8 | 32 | <0.12 | >64 | >64 | 1.54 |
| | | Nitrofurantoin | 8 | 2 | 16 | 4 | 2 | 2 | 4 | >64 | >64 | 1.19 |

Preliminary Antimicrobial Disk Susceptibility Test (Anaerobic Bacteria)

This assessment was performed essentially as described in known literature. [See, NCCLS. Performance Standards for Antimicrobial Disk Susceptibility Tests; Approved Standard-Seventh Edition. NCCLS Document M2-A7. NCCLS: Wayne, Pa.; 2000.]

Medium: Columbia Agar+5% sheep blood (bioMerieux); prereduced in an anaerobic jar. Anaerobic bacterial strains: 1. Clostridium perfringens, 2. Clostridium sp., 3. Bacteroides fragilis, 4. Clostridium species; 5. Clostridium perfringens, fresh isolate from wound swab; and 6. Peptostreptococcus spp., fresh isolate from an abdominal aspirate. Antimicrobial Disks: test compound 200 μg; test compound 80 μg; Nitrofurantoin 200 μg (Becton-Dickinson); Azithromycin 15 μg (Becton-Dickinson); Piperacillin 100 μg (Becton-Dickinson); Ampicillin/Sulbactam 20 μg (Becton-Dickinson).

Procedure: A direct colony suspension method, was performed for inoculum preparation. Test plates were prepared by streaking the swab, which was dipped into the adjusted bacterial suspension, over the entire sterile agar surface. The predetermined battery of antimicrobial disks was dispensed onto the surface of the inoculated agar plates and incubated at 37° C. in an anaerobic jar for 24-48 hours. The diameter of inhibition zones was measured; including the diameter of the disk, and results recorded.

TABLE 3
Antimicrobial Disk Susceptibility Tests for Compound 122

| | ANTIMICROBIAL | INHIBITION |
|---|---|---|
| ANAEROBE STRAIN** | AGENT DISKS | ZONES |
| Clostridium perfringens | Compound 122 200 μg | 36 mm* |
| | Nitrofurantoin 200 μg | >25 mm** |
| Clostridium perfringens | Compound 122 200 μg | 30 mm |
| (fresh wound isolate) | Nitrofurantoin 200 μg | 21 mm |
| Clostridium sp. | Compound 122 200 μg | 20 mm |
| | Nitrofurantoin 200 μg | 15 mm |
| Clostridium sp. | Compound 122 200 μg | 40 mm |
| | Nitrofurantoin 200 μg | >25 mm** |
| Bacteroides fragilis | Compound 122 200 μg | 41 mm |
| | Nitrofurantoin 200 μg | >25 mm** |
| Peptostreptococcus sp | Compound 122 200 μg | 15 mm |
| (Fresh abdominal aspirate | Nitrofurantoin 200 μg | 12 mm** |
| aspirate) | | |

*Zones > 12 mm for Compound 122 considered active
**Inhibition zone overlapped with other inhibition zones

Antimicrobial Agar Dilution Test

This assessment was performed essentially as described in known literature. [See, NCCLS. Methods for Antimicrobial Susceptibility Testing of Anaerobic Bacteria; Approved Standard-Fourth Edition. NCCLS document M 11-A4. NCCLS: Wayne, Pa.; 1997.]

Agar medium: Brucella blood agar supplemented with hemin (5 μg/mL), 5% sheep blood, and vitamin K₁ (1 μg/mL).

Antimicrobial Agents: Standard Antimicrobial Powders (Azithromycin, Chloramphenicol, Nitrofurantoin, Piperacillin, Clindamycin, Penicillin, Imipenem) and test compound, prepared as stock solutions [5120 μg/mL in DMF (dimethylformamide)] and diluted as indicated in Table 3 of the NCCLS Methods for Antimicrobial Susceptibility Testing of Anaerobic bacteria; Approved Standard-Fourth Edition 1997; M11-A4, Vol.17 No 22.

Inoculum Preparation: The test anaerobic strains were selected from enriched Brucella blood agar. Portions of five colonies were directly suspended into Brucella broth medium to achieve a turbidity equivalent to a 0.5 McFarland standard.

Procedure: The medium was prepared according to the manufacturer's directions and distributed into screw-cap tubes. On the day of the test, blood supplement and 2 ml of each concentration of the antimicrobial agent were added to the appropriate tubes of cooled (50° C.) agar. The mixture of media and antimicrobial agent was poured into standard (15×100 mm) round petri dishes and allowed to solidify. A turbidity-adjusted culture of each anaerobic strain was inoculated to each plate by a replicating device (approximately 2 μL per spot). The inoculated plates were incubated at 35° C. in an anaerobic jar. Results were recorded after 48 hours of incubation and expressed as minimum inhibitory concentration (MIC) values. Results are summarized in Table 4.

TABLE 4
ANTIMICROBIAL SUSCEPTIBILITY TESTING OF
ANAEROBIC BACTERIA BY AGAR DILUTION

| | Bacteroides | Bacteroides | Bacteroides | Bacteroides | Bacteroides |
|---|---|---|---|---|---|
| | vulgatus | fragilis | fragilis | fragilis | thetaiotaomicron |
| Compound 122 | <0.125 | <0.125 | <0.125 | <0.125 | <0.125 |
| Nitrofurantoin | <0.125 | 2 | 2 | 2 | 2 |
| Azithromycin | 1 | 4 | 4 | 4 | 4 |
| Chloramphenicol | <0.125 | 4 | 4 | 4 | 4 |
| Piperacillin | 128 | 2 | 16 | 2 | 16 |
| Clindamycin | <0.125 | 1 | 1 | 1 | 0.25 |
| Penicillin | 32 | 4 | 32 | 8 | 8 |
| Imipenem | <0.125 | <0.125 | <0.125 | <0.125 | <0.125 |

In vivo Assays for Antibacterial Activity

The compounds can also be analyzed for antibacterial activity in laboratory animals. These in vivo studies include, but are not limited to, systemic and topical models of infection, urinary tract infection models, helicobacter infections including ulcers of the gastrointestinal tract, sepsis, antibiotic mediated colitis and wound care. The compounds of the invention can also be evaluated in animals to assess their pharmacokinetic profiles, such as oral bioavailability, oral absorption, chemical half-life, identification of metabolites, serum levels at various times, and rate of excretion, for example. The in vivo efficacy data for representative compounds are summarized in Tables 5-7.

Systemic Bacterial Infection Animal Models

Systemic models of infection are described in the literature. The following conditions were used to assay the compounds in this application. Bacteria were grown in Mueller-Hinton agar at 37° C. during 24 h. For each experiment, a bacterial suspension is prepared by inoculating 4-5 bacterial colonies onto Mueller-Hinton broth (MHB) and by incubating at 37° C. for 24 hours to yield approximately 10⁹ CFU/ml. BalbC female were supplied by Charles River. Animals were infected by a single administration of an LD₁₀₀ dose of bacterial culture suspension (1×10⁸ CFU/100 μl per animal) in the tail vein. A careful clinical examination was made several times a day, and obvious clinical symptoms and mortality were recorded. Animals survival was observed for a period of 6 days. Azithromycin was dissolved in 0.5% methocel in saline solution and administered orally. Test compounds were micronized with mortar and pestle and then dissolved in methocel saline solution with 3% of DMF. The first dose was administered 30 minutes after infection, with following doses every 12 hours for 3 days. For compounds administered IP, the dosing schedule was QDx3 (once per day for three days). For compounds given orally (PO) the doses were twice a day.

TABLE 5
Efficacy of Compound 122 administered IP to
mice systemically infected with S. aureus

| | | | | Dose | | Sched- | Survival |
|---|---|---|---|---|---|---|---|
| Study | Infected | Treatment | n | (mg/kg) | Route | ule | (%) |
| #2 | + | Compound 122 | 10 | 100 | IP | QD × 3 | 0 |
| | + | Compound 122 | 10 | 75 | IP | QD × 3 | 10 |
| | + | Compound 122 | 10 | 50 | IP | QD × 3 | 30 |
| | + | Compound 122 | 10 | 25 | IP | QD × 3 | 0 |
| | + | — | 5 | | | QD × 3 | 0 |
| | + | Nitrofurantoin | 10 | 50 | IP | QD × 3 | 50 |
| | + | Nitrofurantoin | 10 | 25 | IP | QD × 3 | 50 |

TABLE 6
Efficacy of Compound 122 administered IP to mice
systemically infected with E. coli

| | | | | Dose | | Sched- | Survival |
|---|---|---|---|---|---|---|---|
| Study | Infected | Treatment | n | (mg/kg) | Route | ule | (%) |
| #3 | + | Compound 122 | 10 | 100 | IP | QD × 3 | 0 |
| | + | Compound 122 | 10 | 75 | IP | QD × 3 | 20 |
| | + | Compound 122 | 10 | 50 | IP | QD × 3 | 60 |
| | + | Compound 122 | 10 | 25 | IP | QD × 3 | 0 |
| | + | — | 5 | | | QD × 3 | 0 |
| | + | Nitrofurantoin | 10 | 100 | IP | QD × 3 | 0 |
| | + | Nitrofurantoin | 10 | 75 | IP | QD × 3 | 20 |
| | + | Nitrofurantoin | 10 | 50 | IP | QD × 3 | 0 |

TABLE 7
Efficacy of Compound 107 administered IP to
mice systemically infected with E. coli

| | | | | Dose | | Sched- | Survival |
|---|---|---|---|---|---|---|---|
| Study | Infected | Treatment | n | (mg/kg) | Route | ule | (%) |
| #4 | + | Compound 107 | 10 | 75 | IP | QD × 3 | 90 |
| | + | Compound 107 | 10 | 50 | IP | QD × 3 | 70 |
| | + | — | 10 | | IP | QD × 3 | 0 |

Assays for Biochemical and Physical-Chemical Properties

The heterocyclic compounds of this invention can be optimized for their in vitro “antibacterial” activity by two types of methods, structural methodology and physical-chemical methodology. The chemical structure can be modified using combinations of substituents to provide compounds which satisfy some or all of the following criteria: 1) a compound in which the calculated or experimentally determined lipophilicity (logP) is in the range of 0 to 2 logP units; 2) a compound which is a substrate for any nitroreductase enzyme; 3) compound in which its redox potential lies within a certain range of about −0.6 and about −0.2, inclusive E₇¹(V); 4) a compound in which its aqueous solubility is greater than 1 ug/mL. These physical-chemical and biochemical properties are contributory factors in the antimicrobial activity of the compounds.

Heterocycles as Nitroreductase Substrates

The antimicrobial mechanism of action of certain nitrofuran-containing compounds like nitrofurazone and furazolidone is mediated by the action of specific oxygen-insensitive nitroreductase enzymes. These include, for example, the gene products NfsA and NfsB from E. coli (Whiteway, J., Koziarz, P., Veall, J., Sandhu, N., Kumar, P., Hoecher, B. and Lambert, I. B. (1998) J. Bacteriol. 180: 5529-5539). In general, molecules in the nitrofuran compound class can be considered pro-drugs, in that they remain relatively benign and inactive until specifically activated by enzymes resident in their target pathogens. Metabolic reduction of the nitrofuran moiety of the administered pro-drug compounds could then lead to the production of reactive intermediates that are subsequently able to damage the internal machinery (proteins, nucleic acids, etc.) of the target organism.

In the past, the powerful structure-based drug design (SBDD) approach to drug discovery and development has been successfully used to guide the design and synthesis of inhibitor drug candidates, whose antagonism of their target enzyme's activity leads to a desired therapeutic outcome (Navia, M. A. and Murcko, M. A. (1992) Curr. Op. Struct. Biol. 2: 202-210). In considering the use of structural methods for design of optimal nitrofuran analog drugs, however, the design principles used in the past can be applied in a novel manner that drives toward an exactly opposite goal, namely an enhancement of the designed substrate prodrug's turnover rate.

In order to apply SBDD to the design of nitrofuran analogs, including those herein, as well as to facilitate an understanding of the structure-activity relations (SAR) observed for these compounds, a model of the major nitroreductase protein NfsA, which is responsible for the activation of nitrofuran drugs in E. coli. and its use is demonstrated herein.

This 3-dimensional construction was initially based on a comparison of the amino acid sequence of the NfsA protein with that of the NADH oxidoreductase Frp (flavin reductase P) from the luminescent bacterium Vibrio harveyi (Zenno, S., Koike, H, Kumar, A. N., Jayaraman, R., Tanokura, M and Saigo, K. (1996) J. Bacteriol. 178: 4508-4514), as shown below in Table 8. This analysis suggested a considerable similarity between the NfsA and Frp proteins, with strong (64%) homology evident in 156 of the 240 residues aligned. Of these, 124 (51%) were identical.

TABLE 8
Table of residues used in nitroreductase-sequence analysis.

Score = 245 bits (625), Expect = 4e-64
Identities = 124/240 (51%), positives = 156/240 (64%)

| | | |
|---|---|---|
| 1 | MNNTIETILAHRSIRKFTAVPITDEQRQTIIQAGLAASSSSMLQVVSIVRVTDSEKRNEL | 60 |
| | M TIE I HRSIR FT PI++ QR+ II + A SSSS LQ SI+R+TD R EL | |
| 1 | MTPTIELICGHRSIRHFTDEPISEAQREAIINSARATSSSSFLQCSSIIRITDKALREEL | 60 |
| 61 | AQFAGNQAYVESAAEFLVFCIDYQRHATINPDVQADFTELTLIGAVDSGIMAQNCLLAAE | 120 |
| | G Q +V AAEF VFC D+ RH I PD Q E L+G VD+ +MAQN L+AAE | |
| 61 | VTLTGGQKHVAQAAEFWVFCADFNRHLQICPDAQLGLAEQLLLGVVDTAMMAQNALIAAE | 120 |
| 121 | SMGLGGVYIGGLRNSAAQVDELLGLPENSAVLFGMCLGHPDQNPEVKPRLPAHVVVHENQ | 180 |
| | S+GLGGVYIGGLRN+ V +LL LP++ LFG+CLG P NP++KPRLPA ++VHEN | |
| 121 | SLGLGGVYIGGLRNNIEAVTKLLKLPQHVLPLFGLCLGWPADNPDLKPRLPASILVHENS | 180 |
| 181 | YQELNLDDIQSYDQTMQAYYASRTSNQKLSTWSQEVTGKLAGESRPHILPYLNSKGLAKR | 240 |
| | YQ L+ + YD+ + YY +R SN + TWS + + ESRP IL YL+ +G A R | |
| 181 | YQPLDKGALAQYDEQLAEYYLTRGSNNRRDTWSHIRRTIIIKESRPFILDYLHKQGWATR | 240 |

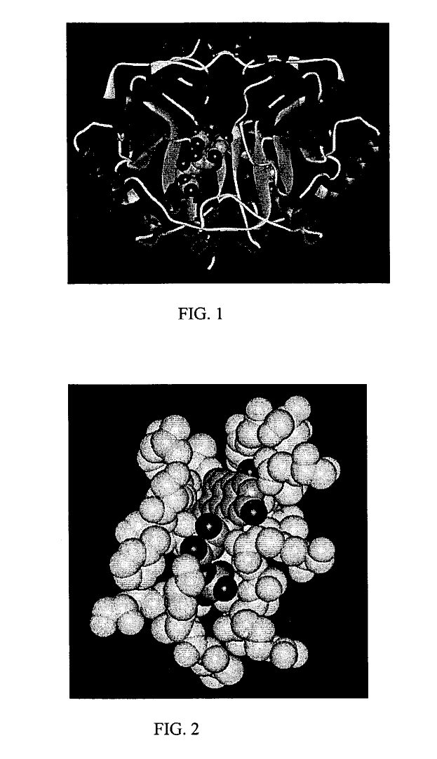

Based on these sequence alignment results, the solved structure (Tanner, J. J., Lei, B., Tu, S. -C. and Krause, K. L. (1996) Biochemistry 35: 13531-13539) of the V. harveyi Frp protein, as shown in FIG. 1, was modified in order to construct a model of the structure of NfsA.

FIG. 2 is a close-up view of the NfsA model structure, which shows the active site of the enzyme. The active site of NfsA is a cavity, exposed to solvent, that incorporates the co-factor FMN (flavin mononucleotide) in a position that is clearly accessible to compounds that might bind to the active site. While not wishing to be bound by theory, one nitroreductase mechanism of action believed to be responsible for the reduction of nitrofuran compounds generally, including those herein, is dependent on the reductive potential of the solvent-accessible cofactor FMN. The oxidized FMN (FMNH₂) that results from the reduction of nitrofuran in this model, is itself reduced to its activated state by the reducing potential of bound NADH, which is readily accommodated by Frp in V. harveyi, as well as by our model of NfsA.

A biochemical assay which measures the reduction turnover rate of a nitroreductase enzyme such as NFsA or NFsB can be used to test heterocyclic compounds as the substrates. Increased enzymatic turnover has been associated to the compound's ability to kill bacteria. Thus, disclosed herein is a model of a nitrofuran reductase which can be used as guide in the design of substrates with high turnover rate. Compounds that dock into the active site and are capable of reduction will provide activated compounds. Compounds that cannot fit into the active site of the nitroreductase model will not be a substrate. Therefore compounds that can be docked into the active site can be used in designing antibacterial analogs.

Using the model of NfsA disclosed herein, one can define classes of nitrofuran analog molecules, including the heterocyclic nitrofurans disclosed herein, that are compatible with the active site of the nitroreductase enzyme, disclosed herein, and which therefore have the potential for metabolic reductive activation, and therefore are useful in discovering new medicinal agents for the treatment of bacterial infections. Modeling techniques are know in the art, including both hardware and software appropriate for creating and utilizing models of receptors and enzyme conformations.

Numerous computer programs are available and suitable for rational drug design and the processes of computer modeling, model building, and computationally identifying, selecting and evaluating potential antimicrobial compounds in the methods described herein. These include, for example, GRID (available form Oxford University, UK), MCSS (available from Molecular Simulations Inc., Burlington, Mass.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Molecular Simulations Inc., Burlington, Mass.), and 3D database systems such as MACCS-3D (available from MDL Information Systems, San Leandro, Calif.), UNITY (available from Tripos, St. Louis. Mo.), and CATALYST (available from Molecular Simulations Inc., Burlington, Mass.). Potential antimicrobial compounds may also be computationally designed “de novo” using such software packages as LUDI (available from Biosym Technologies, San Diego, Calif.), LEGEND (available from Molecular Simulations Inc., Burlington, Mass.), and LEAPFROG (Tripos Associates, St. Louis, Mo.). Compound deformation energy and electrostatic repulsion, may be evaluated using programs such as GAUSSIAN 92, AMBER, QUANTA/CHARMM, AND INSIGHT II/DISCOVER. These computer evaluation and modeling techniques may be performed on any suitable hardware including for example, workstations available from Silicon Graphics, Sun Microsystems, and others. These techniques, methods, hardware and software packages are representative and are not intended to be comprehensive listing. Other modeling techniques known in the art may also be employed in accordance with this invention. See for example, N. C. Cohen, Molecular Modeling in Drug Design, Academic Press (1996) (and references therein), and software identified at internet sites including, for example, the CAOS/CAMM Center Cheminformatics Suite at http://www.caos.kun.nl/, and the National Institutes of Health (“NIH”) Molecular Modeling Home Page at http://www.fi.muni.cz/usr/mejzlik/mirrors/molbio.info.nih.gov/modeling/software_list/.

Models of other nitroreductases present in other target pathogens can be similarly determined based on the known sequence information about those enzymes.

Thus, delineated herein is a strategy to synthesize heterocyclic analogs with greater in vitro potency and superior in vivo efficacy. The initial approach uses calculable physical-chemical parameters to improve the antimicrobial activity of heterocyclic analogs. One calculable parameter that is related to cell-wall penetration through a passive-diffusion process is lipophilicity. Lipophilicity can be measured experimentally or estimated computationally using a variety of different methods. Other physical chemical parameters to be incorporated into this model include reduction potential and aqueous solubility.

Reduction Potential

One physical-chemical, method for evaluating the efficacy of the compounds of the invention is reduction potential. Redox potential has been correlated with the turnover rate of NFsA substrates (Zenno, S., Koike, H, Kumar, A. N., Jayaraman, R., Tanokura, M and Saigo, K. (1996) J. Bacteriol. 178: 4508-4514). The redox potential can be measured experimentally for organic compounds using cyclic voltametry and is known in the art [see, for example, Pletcher, D). Chapter. 6, Electroanalytical Techniques, in A First Course in Electrode Processes, Alresford Press Ltd., 1991.]

Lipophilicity (logP)

A second physical-chemical method for testing compounds of the invention is to calculate or measure the compound's lipophilicity. The lipophilicity (logP) of a compound can be calculated using a variety of methods including: AlogP, clogP, MlogP (Glose, A. K., Viswanadhan, V. N., Wendoloski, J. J., “Prediction of hydrophobic (lipophilic) properties of small organic molecules using fragmental methods: An analysis of AlogP and ClogP methods” J. Phys. Chem. 102, 3762-3772 (1998). Lipophilicity can also be measured experimentally through a variety of methods including the octanol-water shake-flask method or high-performance liquid chromatography (HPLC).

Aqueous Solubility

A third physical-chemical method for testing compounds of the invention (specifically for in vivo activity) is water solubility. The solubility of the compounds of the invention can be measured experimentally using a variety of low- and high-throughput techniques.

Clinical Uses of the Heterocyclic Compounds

The compounds claimed in this invention can be used therapeutically or prophylactically for treatment or prevention of bacterial diseases.

The invention also relates to methods of disrupting the internal regulation of microbial growth or respiration, in a host, comprising the step of administering to said host a compound of any of the formulae described herein or a composition comprising a compound of any of the formulae described herein. In one embodiment, the invention relates to a method of inhibiting microbial or bacterial activity in a host comprising the step of administering to said host a compound, or a composition comprising a compound, of any one of the formulae described herein. Preferably, the host is a human being, or animal.

In an alternate embodiment, this invention relates to a method of treating disease or disease symptoms in a host comprising the step of administering to said host a compound, or a composition comprising a compound, of any of the formulae described herein. Preferably, the host is a human being or animal. The host (or subject) may be one that is in need of such treatment, or one identified as in need of such treatment. The identification may be objective or subjective in nature. For example, by clinical observation (e.g., by a physician or other health care provider), by diagnostic test or screen for the microorganism, or by observation of symptoms or other indicator of the presence of microbial or mycobacterial infection.

Infections and infectious diseases are caused from a variety of microorganisms. The compounds of the invention may find use in the medical treatment of infectious diseases from bacterial sources.

Compounds that kill or limit the growth of microorganisms may find use in the treatment of infections and infectious diseases. Specific bacterial microorganisms are known to be associated with the type of infection or infectious disease. Some examples of bacterial infections and their most common causative pathogens are given below.

Upper and lower respiratory tract infections include, but are not limited to: bronchitis, sinusitis, pneumonia, sore throat, chronic streptococcal infections, diphtheria, acute epiglottitis, influenza, chronic bronchitis, middle ear infections (otitis media), pneumonia, bronchopneumonia, Legionnaire's disease, a typical pneumonia, whooping cough, and tuberculosis.

Bacterial microorganisms causing respiratory tract infections include but are not limited to: S. pyogenes, S. pneumoniae, S. aureus, H. influenzae, M. catarrhalis N. meningitidis, B. pertussis, M. pneumoniae, mycobacteria, including M. tuberculosis, Enterobacteriaceae, Legionella, Anerobes, Nocardia, Pseudomonas, C. psittaci, C. diphtheriae.

Urinary tract infections include, but are not limited to: urethritis, cystitis, pyelonephritis (kidney infection), asymptomatic bacteruria, interstitial cystitis, acute urethral syndrome, and recurrent urinary tract infections.

Bacterial microorganisms causing urinary tract infections include but are not limited to: E. coli, Proteus, Providentia, Pseudomonas, Klebsiella, Enterobacter, Serratia, Coag. neg. Staphylococci, Enterococci, and C. trachomatis.

Skin and Wound Infections include, but are not limited to: erythrasma, panaritium, impetigo, folliculitis, erysipelas, cellulitis, and necrotizing fasciitis.

Bacterial microorganisms causing skin and wound infections include but are not limited to: Staphylococci, P. aeruginosa, P. acnes, Clostridia, anerobes, and B. fragilis.

Bacterial microorganisms causing systemic infections (bacteremia) include but are not limited to: Streptococci, Staphylococci, Enterobacteriaceae, Pseudomonas, Bacteroides sp., Naiseria, H. influenzae, Brucella, Listeria, and Salmonella typhi.

Sexually transmitted diseases of bacterial origin include, but are not limited to: adnexitis, cervicitis, chanchroid, urethritis, balanitis, gonorrhea, lymphogranuloma venereum, syphilis, and granuloma inguinale.

Bacterial microorganisms causing sexually transmitted infections include but are not limited to: Chlamydia, N. gonorrhoeae, U. urealyticum, T. pallidium, G. vaginalis, H. ducreyi, and C. granulomatis, Streptococci, Staphylococci, and Enterobacteriae.

Gastrointestinal infections of bacterial origin include but are not limited to: food borne infections, colitis, enteritis, gastric ulcers, duodenal ulcers, pancreatitis, gall bladder infections, cholera, and thyphus.

Bacterial microorganisms causing gastrointestinal infections include but are not limited to: H. pylori, C. pylori, C. duodeni, S. typhi, S. paratyphi, V. cholerae, anaerobes, enterobacteriaceae, Staphylococci, and Streptococci.

Methods of Treating Patients

The heterocyclic compounds of the formulae delineated herein can be administered to a patient, for example, in order to treat an infection such as a bacterial infection. The heterocyclic compounds can, for example, be administered in a pharmaceutically acceptable carrier such as physiological saline, in combination with other drugs, and/or together with appropriate excipients. The heterocyclic compounds of the formulae herein can, for example, be administered by injection, intravenously, intraarterially, subdermally, intraperitoneally, intramuscularly, or subcutaneously; or orally, buccally, nasally, transmucosally, topically, in an ophthalmic or otic preparation, or by inhalation, with a dosage ranging from about 0.001 to about 100 mg/kg of body weight, preferably dosages between 10 mg and 5000 mg/dose, every 4 to 120 hours, or according to the requirements of the particular drug.

As the skilled artisan will appreciate, lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

In an alternate embodiment, this invention provides methods of treating, preventing, or relieving symptoms of disease in a mammal comprising the step of administrating to said mammal any of the pharmaceutical compositions and combinations described above. Preferably, the mammal is a human. If the pharmaceutical composition only comprises the compound of this invention as the active component, such methods may additionally comprise the step of administering to said mammal an additional therapeutic agent such as, for example, macrolide antibiotics (e.g., clarithromycin), proton pump inhibitors (e.g., omeprazole), rifamycins (e.g., rifampin), aminoglycosides (e.g., streptomycin, gentamycin, tobramycin), penicillins (e.g., penicillin G, penicillin V, ticarcillin), β-lactamase inhibitors, cephalosporins (e.g., cefazolin, cefaclor, ceftazidime), and antimycobacterial agents (e.g., isoniazid, ethambutol, rifampin). Other suitable agents are delineated in infectious disease texts and publications, including for example, Principles and Practice of Infectious Diseases, G. L. Mandell et al. eds., 3^{rd} ed., Churchhill Livingstone, N.Y., (1990). Such additional agent(s) may be administered to the mammal prior to, concurrently with, or following the administration of the composition having a compound of any of the formulae herein.

Pharmaceutical compositions of this invention comprise a compound of the formulae described herein or a pharmaceutically acceptable salt thereof; an additional agent selected from an anticancer agent, an anti-viral agent, antifungal agent, proton pump inhibitor, antibiotic, and any pharmaceutically acceptable carrier, adjuvant or vehicle. Alternate compositions of this invention comprise a compound of the formulae described herein or a pharmaceutically acceptable salt thereof; and a pharmaceutically acceptable carrier, adjuvant or vehicle. Such compositions may optionally comprise additional therapeutic agents, including, for example an additional agent selected from an anticancer agent, an anti-microbial agent, an anti-viral agent, antifungal agent, proton pump inhibitor, or antibiotic. The compositions delineated herein include the compounds of the formulae delineated herein, as well as additional therapeutic agents if present, in amounts effective for achieving a modulation of microbial or bacterial levels.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration, inhalation, or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrastemal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase combined with emulsifying and/or suspending agents. If desired, certain sweetening and/or flavoring and/or coloring agents may be added.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically applied transdermal patches are also included in this invention.

The compounds and compositions of this invention are useful as sterilizants, antiseptics, adjuvants in wound dressings (e.g., bandages), and adjuvants in wound cleansing methods (swipes, gavage, etc.).

Dosage levels of between about 0.01 and about 100 mg/kg body weight per day, alternatively between about 0.5 and about 75 mg/kg body weight per day of the antimicrobial compounds described herein are useful in a monotherapy and/or in combination therapy for the prevention and treatment of microbial mediated disease. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

When the compositions of this invention comprise a combination of a compound of the formulae described herein and one or more additional therapeutic or prophylactic agents, both the compound and the additional agent should be present at dosage levels of between about 10 to 100%, and more preferably between about 10 to 80% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Examples of potential application of combination therapies include compounds of any of the formulae delineated herein and the following: in combination with a macrolide antibiotic and a proton pump inhibitor for the treatment of gastritis and associated diseases caused by Helicobacter pylori.; in combination with antibacterials such as ciprofloxacin and amoxicillin for the treatment of urinary tract infections; in combination with rifamycins for treatment of staphylococcal infections; in combination with rifamycins, isoniazid, ethambutol, or aminoglycosides for the treatment of mycobacterial infections; in combination with ticarcillin, gentamycin or tobramycin for the initial treatment of neutropenic patients with presumed infections, in combination with penicillins or aminoglycosides for the treatment of enterococcal endocarditis; in combination with antibiotics for the treatment of intraperitoneal, pelvic or other polymicrobial infections.

Other Uses

In another embodiment, the compounds described herein are useful for crystallizing or co-crystallizing with a protein. Such crystals or crystal complexes may additionally comprise additional peptides and or metal ions. The crystals or crystal complexes may be used for investigation and determination of enzyme characteristics including, for example, structure of a microbial regulating enzyme, enzyme active site domains, and ligand-enzyme interactions. This information is useful in developing compounds with modified characteristics and for understanding structure-function relationships of the enzymes and their enzyme-ligand interactions.

In an alternate embodiment, the inhibitory compounds described herein may be used as platforms or scaffolds which may be utilized in combinatorial chemistry techniques for preparation of derivatives and/or chemical libraries of compounds. Such derivatives and libraries of compounds have antimicrobial activity and are useful for identifying and designing compounds possessing antimicrobial activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60. Thus, one embodiment relates to a method of using the compounds described in the formulae herein for generating derivatives or chemical libraries comprising: 1) providing a body comprising a plurality of wells; 2) providing one or more compounds of the formulae described herein in each well; 3) providing an additional one or more chemicals in each well; 4) isolating the resulting one or more products from each well. An alternate embodiment relates to a method of using the compounds described in the formulae herein for generating derivatives or chemical libraries comprising: 1) providing one or more compounds of the formulae described herein attached to a solid support; 2) treating the one or more compounds of the formulae described herein attached to a solid support with one or more additional chemicals; 3) isolating the resulting one or more products from the solid support. In the methods described above, “tags” or identifier or labeling moieties may be attached to and/or detached from the compounds of the formulae herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms (see illustration), in such instances, the

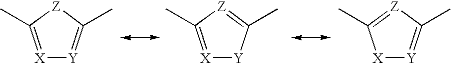

invention expressly includes all tautomeric forms of the compounds described herein (e.g., alkylation of a ring system may result in alkyation at multiple sites, the invention expressly includes all such reaction products). All such isomeric forms of such compounds are expressly included in the present invention. All crystal forms of the compounds described herein are expressly included in the present invention.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following examples. It should be understood that these examples are for illustrative purposes only and are not to be construed as limiting this invention in any manner.

### EXAMPLES

Liquid chromatographic data was obtained using a Hewlett-Packard (HP) 1090 Series Liquid Chromatograph coupled to a Diode Array Detector [Restek Allure C18 Column; particle size, 5 μM; column length, 150 mm; column diameter, 4.6 mm; flow rate, 1 mL/min; Solvent program, from 95% H₂O (w/0.1% TFA)/5% CH₃CN (w/0.1% TFA) to 100% CH₃CN (w/0.1% TFA) in 8 minutes, then held constant for 3 minutes; detection wavelength, 254 nm]. ¹H- and ¹³C-NMR spectra were obtained on a Bruker AC-300 MHz instrument. Medium pressure flash chromatography was performed on an Isco Inc., Combiflash Sg100c system. Thin-layer chromatography was performed using EM Science silica gel 60 F₂₅₄ plastic TLC plates. Melting points were determined in open-air capillary tubes in a Meltemp II apparatus. UV light was used for detecting compounds on the TLC plates. Reagents used in reactions were purchased from the Aldrich Chemical Co. (Milwaukee, Wis.), Sigma Chemical Co. (Saint Louis, Mo.), Fluka Chemical Corp. (Milwaukee, Wis.), Fisher Scientific (Pittsburgh, Pa.), TCI America (Portland, Oreg.), Transworld Chemicals, Inc. (Rockville, Md.), Maybridge Chemical Ltd., (London, England) or Lancaster Synthesis (Windham, N.H.).

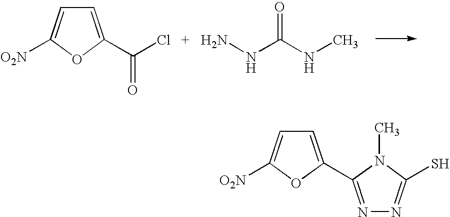

### Example 1

### 5-[2-(5-Nitro)furyl]-4-methyl-4H-1,2,4-triazole-3-thiol (Compound 122)

A stirred suspension of 4-methyl-3-thiosemicarbazide (2.10 g, 20 mmol) in dry methylene chloride (150 mL) was cooled to 0° C. under argon. Pyridine (2.01 mL, 25 mmol) was added dropwise to the reaction mixture and the mixture was stirred for 5 min. A solution of 5-nitro-2-furoyl chloride (3.50 g, 20 mmol) in dry methylene chloride (100 mL) was added dropwise at 0° C. to the above solution. The reaction mixture was stirred and slowly warmed to room temperature. It was monitored by HPLC and the reaction was complete in 4.5 hr. Hydrochloric acid (10% aqueous, 20 mL) was added to the reaction mixture. The resulting solid was filtered through fritted glass, dried under vacuum to yield the intermediate compound (3.82 g, 78.3%, HPLC Rt=4.7 min), which was suspended in water (90 mL) and 0.1M sodium bicarbonate solution (135 mL). The reaction was refluxed under argon, and monitored by HPLC. The reaction was complete in 90 min. The reaction mixture was then cooled and neutralized by 10% HCl to pH=5 (paper). A reddish brown solid precipitated. The solid was collected on a filter and dried under vacuum. This material was recrystallized from ethanol to yield (3.21 g, 71.2%): HPLC Rt=5.70 min; MS m/z=226 (M−1); 1H-NMR (CDCl₃+CD₃OD) δ 3.88(s, 3H), 7.10(d, 1H), 7.40(d, 1H).

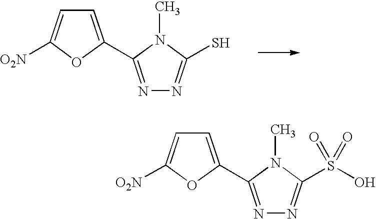

### Example 2

### 5-[2-(5-Nitro)furyl]-4-methyl-4H-1,2,4-triazole-3-sulfonic Acid (Compound 107).

Essentially following a general literature procedure for the preparation of sulfonic acids from mercaptans (Irako, N., et al., Tetrahedron Lett. 1998, 39, 5793-5796) to a stirred solution of 5-[2-(5-nitro)furyl]-4-methyl-4H-1,2,4-triazole-3-thiol (Compound 122) (909 mg, 4 mmol) in trifluoroacetic acid (4 mL) was added hydrogen peroxide (50% aqueous solution, 5 mL) dropwise under argon. The reaction mixture was stirred and monitored by HPLC. The reaction was complete in 1 hr. The solvent was then removed under reduced pressure and the crude compound was recrystallized from methanol to yield a mixture, including Compound 107 (820 mg, 74.8%): HPLC Rt=4.556 min (100%); MS m/z 273(M−1). Variations in reaction temperature and amount of hydrogen peroxide used resulted in a variation of the mixture component ratios observed.

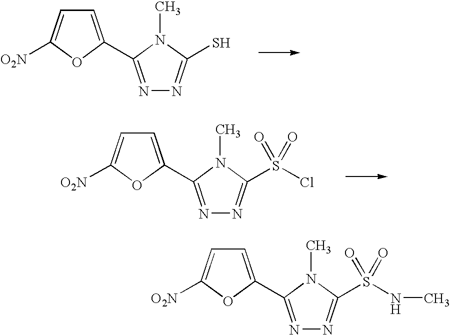

### Example 3

### 5-[2-(5-Nitro)furyl]-4-methyl-4H-1,2,4-triazole-3-methyl Sulfonamide (Compound 106)

Essentially following a general literature procedure for the synthesis of sulfonyl chlorides from mercaptans (Vedejs, E., et al., J. Org. Chem. 2000, 65, 2309-2318) a stirred solution of 33% aqueous acetic acid (5 mL) was cooled to 0-5° C. (ice-salt mixture). Chlorine gas was passed through the reaction mixture until a yellow precipitate formed. A suspension of 5-[2-(5-nitro)furyl]-4-methyl-4H-1,2,4-triazole-3-thiol (Compound 122) (560 mg, 2.5 mmol) in acetic acid (33% aqueous, 5 mL) was added dropwise while the internal temperature maintained at 0-5° C. The yellow suspension was stirred for additional 15 min. The reaction mixture was then diluted with cold methylene chloride (120 mL). The resulting organic layer was washed with cold saturated sodium bicarbonate solution (20 mL) and cold brine (15 mL). The organic layer was dried over sodium sulfate at −10° C., filtered through celite, and concentrated under reduced pressure until a thick slurry formed. Dry diethyl ether (15 mL) was added, the mixture was cooled in dry ice-acetone bath, and collected by suction filtration. The fluffy solid was washed with diethyl ether and dried under vacuum. The compound was used in the following reactions without further purification.

Essentially following a general literature procedure for the synthesis of sulfonamides from sulfonyl chlorides (Grunewald, G. L., et al., J. Med. Chem. 1999, 42, 1982-1990) to a stirred solution of sulfonyl chloride (29 mg, 0.1 mmol) in dry THF (15 mL) was added methyl amine (2 M solution in THF, 30 mL) at 0° C. under argon. The reaction was refluxed 18 h. The solvent was then removed and the compound was dried under vacuum to yield Compound 106 (22 mg, 76.9%): HPLC Rt=5.662 min (100%); MS m/z 286 (M−1).

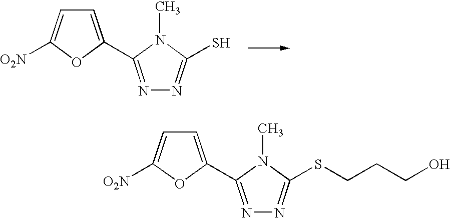

### Example 4

### 5-[2-(5-Nitro)furyl]-4-methyl-4H-1,2,4-triazole-3-(3-hydroxypropyl)-thiol (Compound 116)

To a stirred solution of 5-[2-(5-nitro)furyl]-4-methyl-4H-1,2,4-triazole-3-thiol (Compound 122) (909 mg, 4 mmol) in acetone (120 mL) was added potassium carbonate (5.00 g, 36.2 mmol) at 0° C. under argon. The reaction mixture was stirred for 2 min and a solution of 3-bromo-1-propanol (2.50 g, 18 mmol) in acetone (20 mL) was added dropwise at 0° C. The reaction mixture was warmed to room temperature and then refluxed under argon. The reaction was monitored by HPLC and was complete in 4 hr. The reaction mixture was then filtered, the solvent removed under vacuum, and crude product was purified by flash chromatography using 5-15% methanol in methylene chloride as solvent to yield Compound 116 (950 mg, 83.6%): HPLC Rt=5.02 min (100%); MS m/z 283 (M−1); ¹H-NMR (CDCl₃+CD₃OD) δ 2.01 (m, 2H), 3.50 (m, 2H), 3.80 (m, 2H), 3.90 (s, 3H), 7.22 (d, 1H), 7.40 (d, 1H).

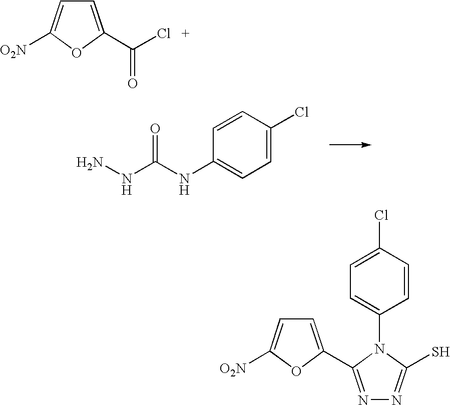

### Example 5

### 4-(4-Chlorophenyl)-5-(5-nitrofuranyl)-4H-1,2,4-triazol-3-thiol (Compound 139)

To a stirred solution of 4-chlorophenyl-3-thiosemicarbazide (4.22 g, 21 mmol) in dry methylene chloride (150 ml) at 0° C. was added a solution of 5-nitro-2-furoyl chloride (3.50 g, 20 mmol) in dry methylene chloride (30 mL) dropwise under an inert atmosphere. The reaction mixture was slowly warmed to room temperature and stirred for 48 hours. The reaction mixture was then filtered. The solvent was removed from the mother liquor, and the crude product was purified by combiflash chromatography using 0-55% ethyl acetate in hexane over 40 minutes. Fractions were monitored by HPLC. The desired product came off the HPLC at 7.8 minutes. Other peaks formed at 8.3, 8.0, 7.2, and 5 minutes. The solvent was removed, and the product was then dried under vacuum. The yield of Compound 139 was 600 mg (9.3%). The following analytical data was obtained: MS-321 (M−H⁻); NMR (CDCl₃), 6.60 (dd, 1H), 7.15 (dd, 1H), 7.30 (dd 1H), 7.40 (dq, 2H), 7.60 (dd, 1H).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of the formula: 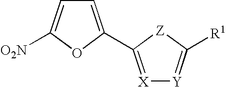
wherein,
Each X is independently selected from CR², NR², O, S, or N;
Each Y is independently selected from CR², NR², O, S, or N;
Each Z is independently selected from CR², NR², O, S, or N;
Each R¹ is independently selected from SH, SO₃H, SO₂NH₂, SO₂NR³R³, S(CH₂)nOH, OR³, S(CH₂)nOC(O)R³, or SR³;
Each n is independently 1, 2 or 3;
Each R² is independently selected from H, C1-C10 alkyl; C2-C10 alkenyl; C2-C10 alkynyl; C3-C10 cycloalkyl; aryl optionally substituted with 1-4 independent R⁵; heteroaryl optionally substituted with 1-4 independent R⁵; heterocyclyl optionally substituted with 1-4 independent R⁵; and C1-C10 alkyl substituted with R⁴ or R⁸;
Each R³ is independently selected from H, C1-C10 alkyl; C2-C10 alkenyl; C2-C10 alkynyl; C3-C10 cycloalkyl; aryl optionally substituted with 1-4 independent R⁵; heteroaryl optionally substituted with 1-4 independent R⁵; heterocyclyl optionally substituted with 1-4 independent R⁵; and C1-C10 alkyl substituted with R⁴ or R⁸;
Each R⁴ is independently selected from halogen, CF₃, SR⁶, OR⁶, OC(O)R, NR⁶R⁶, NR⁶R⁷, COOR⁶, NO₂, CN, C(O)R⁶, or C(O)NR⁶R⁶;
Each R⁵ is independently selected from C1-C10 alkyl; C2-C10 alkenyl; C2-C10 alkynyl; C3-C10 cycloalkyl; C4-C10 cycloalkenyl; halo; haloalkyl; SR⁶; OR⁶; NR⁶R⁶; NR⁶R⁷; COOR⁶; NO₂; CN; C(O)R⁶; C(O)NR⁶R⁶; OC(O)R⁶; S(O)₂R⁶; S(O)₂NR⁶R⁶; NR⁶C(O)N R⁶R⁶; NR⁶C(O)R⁶; NR⁶(COOR⁶); NR⁶C(O)R⁸; NR⁶S(O)₂NR⁶R⁶; NR⁶S(O)₂R⁶; NR⁶S(O)₂R⁸; and C1-C10 alkyl substituted with R⁴ or R⁸;
Each R⁶ is independently selected from H, C1-C10 alkyl; C2-C10 alkenyl; C2-C10 alkynyl; C3-C10 cycloalkyl; R⁸; and C1-C10 alkyl substituted with R⁸ or R¹⁰;
Each R⁷ is independently selected from C(O)R⁹, COOR⁹;
Each R⁸ is independently selected from aryl, heteroaryl or heterocyclyl, each optionally substituted with 1-4 independent R¹⁰;
Each R⁹ is independently C1-C10 alkyl, aryl, heteroaryl, or heterocyclyl, each optionally substituted with 1-4 independent R¹⁰;
Each R¹⁰ is independently halogen, CF₃, SR¹¹, OR¹¹, OC(O)R¹¹, NR¹¹R¹¹, NR¹¹R⁷, COOR¹¹, NO₂, CN, C(O)R¹¹, or C(O)NR¹¹R¹¹;
Each R¹¹ is independently selected from H, C1-C10 alkyl or aryl, each optionally substituted with halogen, SR¹², OR¹², OC(O)R¹², or NR¹²R¹²;
Each R¹² is independently selected from H, C1-C10 alkyl, heteroaryl, heterocyclyl, or aryl;
Each aryl is independently a 6-carbon monocyclic or 10-carbon bicyclic aromatic ring system wherein 0, 1, 2 or 3 atoms of each ring may be substituted by a substituent;
Each heteroaryl is independently an aromatic 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent; and
Each heterocyclyl is independently a nonaromatic 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent;
or pharmaceutically acceptable salt thereof.

2. The compound of claim 1, wherein each R¹ is independently selected from SO₃H, SO₂NH₂, SO₂NR³R³, S(CH₂)nOH, or S(CH₂)nOC(O)R³.

3. The compound of claim 1, wherein each R¹ is independently selected from SO₃H, SO₂NH₂, or SO₂NR³R³.

4. The compound of claim 1 wherein,
Each Z is independently NR²; and
Each R² is independently selected from C2-C10 alkyl; C2-C10 alkenyl; C2-C10 alkynyl; C3-C10 cycloalkyl; aryl optionally substituted with 1-4 independent R⁵; heteroaryl optionally substituted with 1-4 independent R⁵; heterocyclyl optionally substituted with 1-4 independent R⁵; or C1-C10 alkyl substituted with R⁴ or R⁸; and
Each R⁴ is independently selected from halogen, CF₃, SR⁶, OR⁶, OC(O)R⁶, NR⁶R⁶, NR⁶R⁷, NO₂, CN, C(O)R⁶, or C(O)NR⁶R⁶.

5. The compound of claim 1 wherein,
Each Z is independently NR²; and
Each R² is independently aryl optionally substituted with 1-4 independent R⁵; heteroaryl optionally substituted with 1-4 independent R⁵; or heterocyclyl optionally substituted with 1-4 independent R⁵.

6. The compound of claim 1 wherein,
Each Z is independently NR²; and
Each R² is independently selected from C1-C10 alkyl substituted with R⁴ or R⁸; and
Each R⁴ is independently selected from halogen, CF₃, SR⁶, OR⁶, OC(O)R⁶, NR⁶R⁶, NR⁶R⁷, NO₂, CN, C(O)R⁶, or C(O)NR⁶R⁶.

7. The compound of claim 1, wherein
Each Z is independently NR²; and
Each R² is independently aryl optionally substituted with 1-4 independent R⁵.

8. The compound of claim 1, wherein X and Y are both N.

9. The compound of claim 1, wherein X and Y are not identical.

10. The compound of claim 1, wherein X and Y are one of the following:
X is CR² and Y is N; or
X is N and Y is CR².

11. The compound of claim 1, wherein X, Y and Z are one of the following:
X is CR², Y is N, and Z is NR²;
X is N, Y is CR², and Z is NR²;
X is N, Y is N, and Z is NR²;
X is CR²,Y is N, and Z is O;
X is N, Y is CR², and Z is O;
X is CR², Y is N, and Z is S; or
X is N, Y is CR², and Z is S.

12. The compound of claim 1 wherein,
Each R² is independently selected from aryl optionally substituted with 1-4 independent R⁵; heteroaryl optionally substituted with 1-4 independent R⁵; heterocyclyl optionally substituted with 1-4 independent R⁵; and C1-C10 alkyl substituted with R⁴ or R⁸.

13. The compound of claim 1 wherein,
Each R² is independently selected from aryl optionally substituted with 1-4 independent R⁵; or heteroaryl optionally substituted with 1-4 independent R⁵.

14. A composition comprising a compound of any of claims 1-13 and a pharmaceutically acceptable carrier.

15. A composition comprising a compound of any of claims 1-13, an additional therapeutic agent, and a pharmaceutically acceptable carrier.

16. A composition comprising a compound of any of claims 1-13, an additional therapeutic agent, and a pharmaceutically acceptable carrier, wherein the additional therapeutic agent is an antibacterial agent.

17. A method of treating a subject infected with one or more bacteria, comprising administering to the subject an effective amount of a compound of any of claims 1-13.

18. A method for treating infection in a subject comprising administration of a composition comprising a compound of any of claims 1-13.

19. A method of making a compound of claim 1 comprising taking a 5-nitrofuran-2-carbonylchloride and reacting it with one or more chemical reagents in one or more steps to produce a compound of claim 1.

20. The method of claim 19 comprising taking any one of the intermediate compounds made from 5-nitrofuran-2-carbonylchloride and reacting it with one or chemical reagents in one or more steps to produce a compound of claim 1.

21. A method for identifying a compound having antibacterial activity comprising:
a) assessing the structure of a compound of any of claims 1-13;
b) procuring a derivative compound of the compound in step a);
c) assessing the antibacterial activity of the derivative compound.

22. A method for identifying a compound having antibacterial activity comprising:
a) taking a candidate compound;
b) assessing the binding affinity of the candidate compound in a model of the nitroreductase enzyme NFSA;
c) assessing the antibacterial activity of the candidate compound.

23. A compound of any of claims 1-13, having one or more of the following properties:
a) a compound in which the calculated or experimentally determined lipophilicity (logP) is in the range of 0 to 2 logP units;
b) a compound which is a substrate for any nitroreductase enzyme;
c) a compound having a redox potential between about −0.6 and about −0.2, inclusive E71(V);
d) a compound having aqueous solubility greater than 1 μg/mL.

24. A formulation comprising a compound of any of claims 1-13, and an excipient suitable for administration to a subject.

25. A method of treating a subject having a microbial infection comprising administering to the subject an effective amount of a formulation of claim 24.

26. The method of claim 25, wherein the subject is a human.

27. The method of claim 25, wherein the subject is an animal.

28. A method of inhibiting bacterial growth in a non-living system comprising contacting the system with an effective amount of a compound of any of claims 1-13.

29. A method of treating tuberculosis in a subject in need of treatment thereof, comprising administering to the subject an effective amount of a compound of any of claims 1-13.

30. A method of prophylaxis of a subject infected with one or more bacteria, comprising administering to the subject an effective amount of a compound of any of claims 1-13.

31. A method for prophylaxis of infection in a subject comprising administration of a composition of claim 14.
